(19) United States
(12) Patent Application Publication
(10) Pub. No.: US 2011/0005517 A1
(43) Pub. Date: Jan. 13, 2011

(54) ATOMIZER, AND FILTER

(75) Inventors: Georg Boeck, Laupheim (DE); Achim Moser, Chemnitz (DE); Klaus Kadel, Witten (DE)

Correspondence Address: MICHAEL P. MORRIS;BOEHRINGER INGELHEIM USA CORPORATION, 900 RIDGEBURY ROAD, RIDGEFIELD, CT, 06877-0368, US

(73) Assignee: BOEHRINGER INGELHEIM PHARMA GMBH & CO. KG, Ingelheim (DE)

(21) Appl. No.: 12/599,717

(22) PCT Filed: May 13, 2008
(86) PCT No.: PCT/EP08/55863

§ 371 Date: Aug. 26, 2010

(30) Foreign Application Priority Data
May 15, 2007 (DE) 10 2007 023 012.7

(51) Int. Cl. A61M 11/00; B23P 17/04; B01D 39/00

(52) U.S. Cl. 128/200.23; 29/428; 210/501

(57) ABSTRACT

Disclosed are an atomizer for a fluid, especially for medical aerosol therapy, and a filter for said atomizer in order to prevent germs from spreading. The atomizer comprises a protective device for preventing germs from infesting the preferably preservative-free fluid. Particularly the filter can be used as a protective device. This prevents germs from infesting the fluid during the service life of the atomizer even when the atomizer is used several times.

The present invention relates to an atomizer according to the preamble of claim 1, a filter according to the preamble of claim 22 and methods for producing the atomizer.

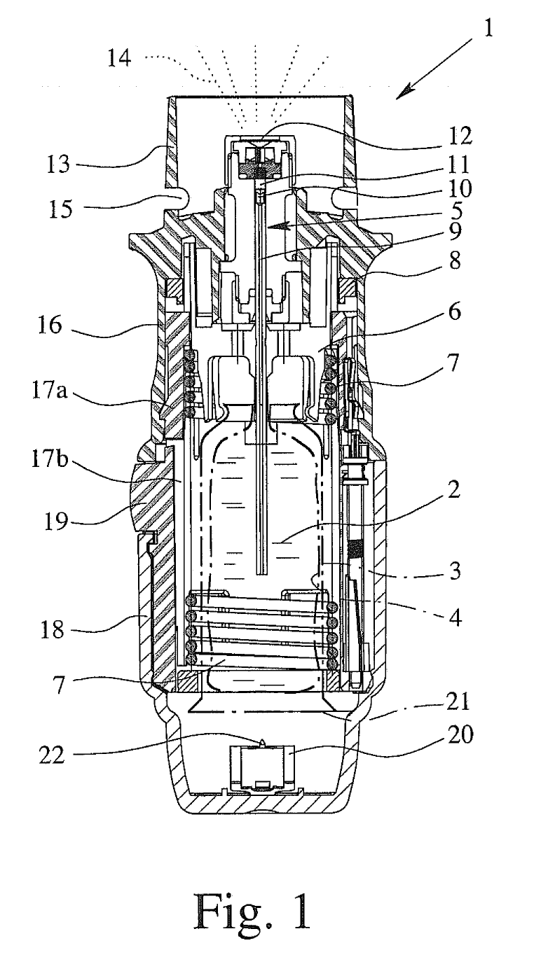

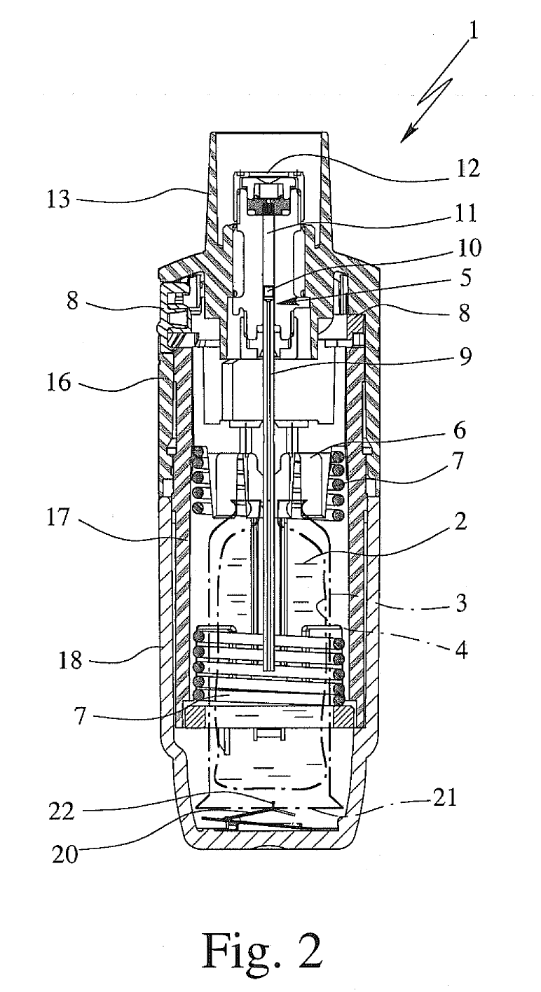

The starting point of the present invention is an atomizer that is marketed under the trade mark RESPIMAT® in the form of an inhaler, as shown in principle in WO 91/14468 A1 and specifically in WO 97/12687 A1 (FIG. 6a, 6b) and in FIGS. 1 and 2 of the attached drawings. The atomizer comprises as reservoir for a fluid that is to be to atomized a rigid insertable container having an inner bag containing the fluid and a pressure generator with a drive spring for conveying and atomizing the fluid.

A particular problem when the atomizer is used a number of times is that the fluid may become contaminated with germs over time. Up till now, preservatives have normally been used to prevent the contamination. However, because of increasing allergies, intolerances and incompatibilities with active substances or adjuvants or with components of the atomizer or the like, it is desirable to stop using preservatives.

The problem of the present invention is to provide an atomizer, a filter for a fluid that is to be atomized and methods of producing the atomizer, while avoiding or at least reducing the use of preservatives and/or contamination of the fluid that is to be atomized.

The above problem is solved by an atomizer according to claim 1, a filter according to claim 22 or a method according to claim 34 or 36. Advantageous further features are the subject of the subclaims.

In one aspect the present invention proposes providing the atomizer with a protective device to prevent contamination of the fluid and/or using a fluid that is free from preservatives and/or at least substantially free from preservatives.

The protective device may be a germ-proof, in particular sterile wrapper such as a bag for holding the atomizer and preferably the container ready for delivery.

The protective device may be formed by the atomizer constituting or comprising a germ-proof, in particular sterile receiving space for the container, particularly a housing part that can be fitted on in germ-proof manner.

Particularly preferably, the container, which is initially still sealed in its delivered to state, is already mounted in the atomizer in the delivered state. The container can then be opened immediately before use of the atomizer, for example by pushing a housing part, or not until the atomizer is used for the first time.

Alternatively or additionally, the protective device may have a contact portion for the fluid that kills bacteria or other germs, for example made of silver and/or copper or activated charcoal.

Alternatively or additionally, the protective device may have a lockable valve for preventing the contamination of the fluid in the container, in a conveying element and/or in a pressure generator in the atomizer.

Alternatively or additionally, the atomizer and/or container may have an at least substantially germ-proof filter as protective device.

The above measures may be adopted alternatively or in any desired combination to prevent or at least minimise the possible germ contamination of the fluid in the container and/or in the atomizer. In particular, the use of preservatives in the fluid can then be avoided or at least reduced.

In another aspect, the invention proposes using a filter for a fluid that is to be atomized or dispensed by some other method, particularly for medical aerosol therapy or for other, particularly medical, therapeutic or preventive purposes, while the openings in the filter being sufficiently small to make the filter substantially germ-proof. This is a simple way of effectively improving the protection against contamination.

The proposed filter is preferably used in an atomizer or inhaler. However, the filter may also be used for other purposes.

Further advantages, features, properties and aspects of the present invention will become apparent from the claims and the following description of preferred embodiments with reference to the drawings, wherein:

FIG. 1 shows a schematic section through a known atomizer in the non-tensioned state;

FIG. 2 shows a schematic section, rotated through 90° compared with FIG. 1, through the known atomizer in the tensioned state;

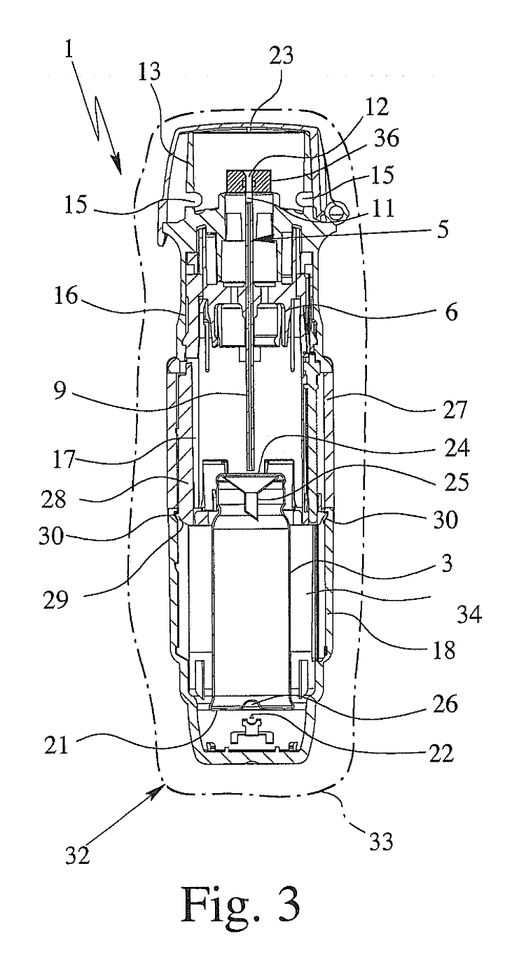

FIG. 3 shows a schematic section through a proposed atomizer according to a first embodiment in the delivered state with the sealed container incorporated therein;

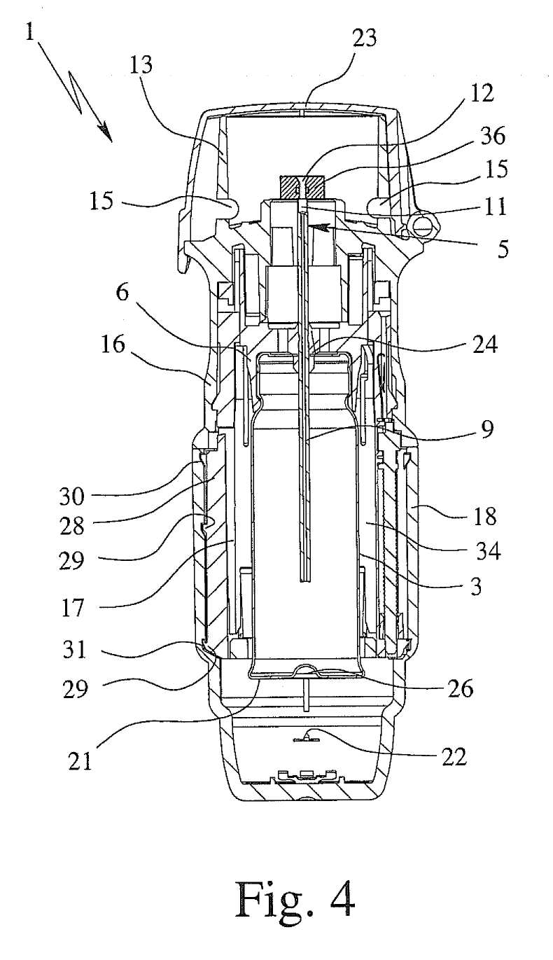

FIG. 4 shows a schematic section through the atomizer according to FIG. 3 in the activated state or with the container opened;

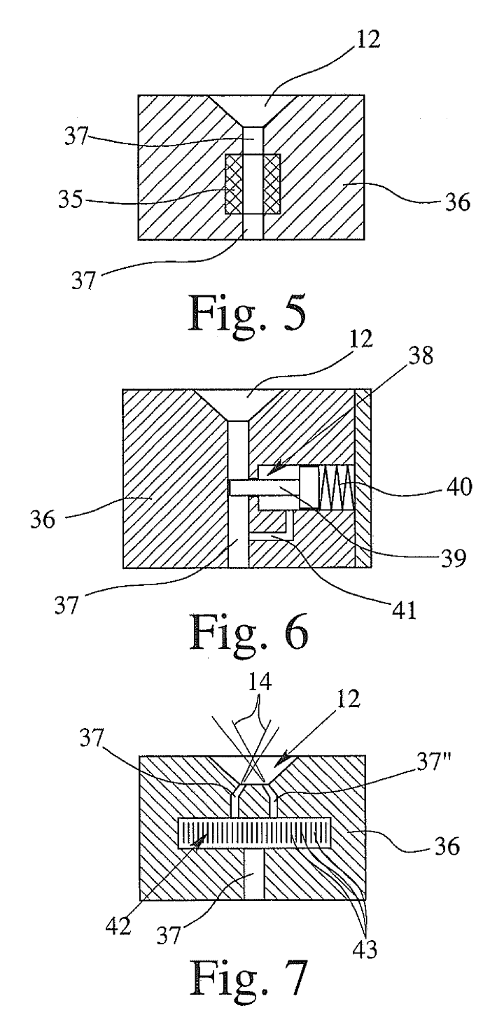

FIG. 5 shows a schematic section through a part of the atomizer according to FIG. 3;

FIG. 6 shows a section corresponding to FIG. 5 through a second embodiment of the atomizer;

FIG. 7 shows a section corresponding to FIG. 5 through a third embodiment of the atomizer;

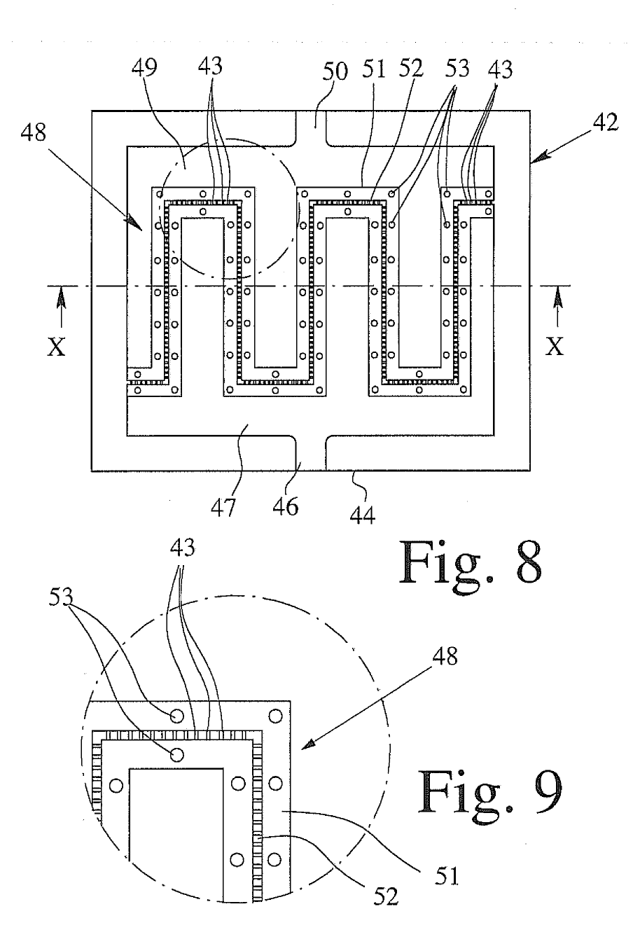

FIG. 8 shows a schematic plan view of a proposed filter in the opened state;

FIG. 9 shows a magnified detail from FIG. 8;

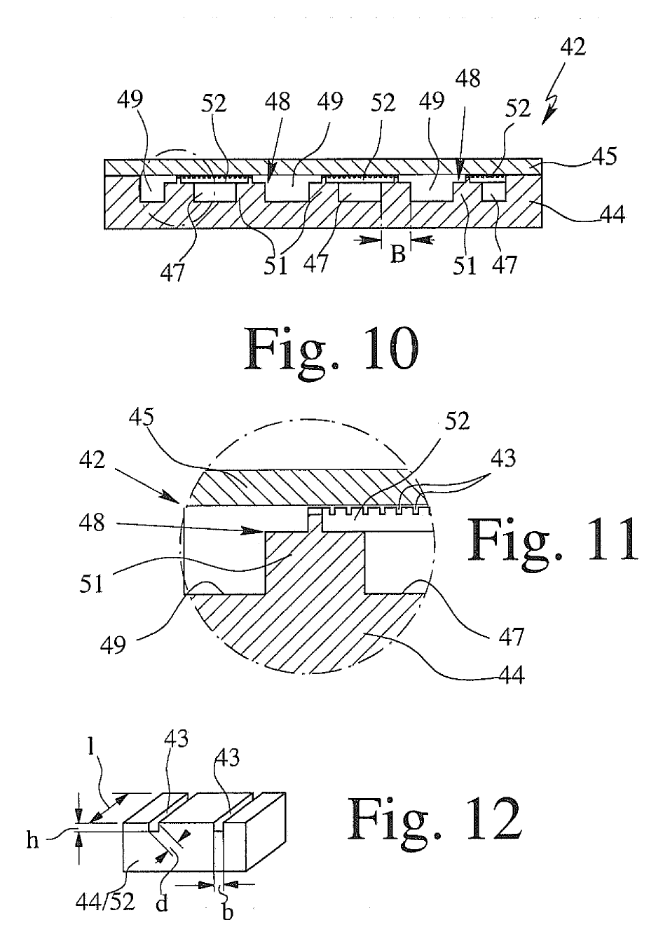

FIG. 10 shows a section through the filter along the line X-X in FIG. 8, showing the filter in the closed state;

FIG. 11 shows a magnified detail from FIG. 10; and

FIG. 12 shows a perspective view of a detail of the open filter according to FIG. 8.

In the Figures the same reference numerals have been used for identical or similar parts, and corresponding or comparable properties and advantages are achieved even if the associated description is not repeated.

FIGS. 1 and 2 show a known atomizer 1 for atomizing a fluid 2, particularly a highly effective medicament or the like, shown schematically in the non-tensioned state (FIG. 1) and in the tensioned state (FIG. 2). The atomizer 1 is embodied in particular as a portable inhaler and preferably operates without propellant gas.

Atomization of the fluid 2, preferably a liquid, more particularly a medicament, produces an aerosol 14 preferably destined for the lungs (FIG. 1) which can be breathed in or inhaled by a user or patient (not shown). Normally, the medicament is inhaled at least once a day, more particularly several times a day, preferably at predetermined intervals, depending on the ailment affecting the patient.

The known atomizer 1 has a preferably insertable and in particular replaceable container 3 containing the fluid 2. The container 3 thus forms a reservoir for the fluid 2 which is to be atomized. Preferably, the container 3 contains a sufficient amount of fluid 2 or active substance to provide, for example, up to 200 dosage units or doses, i.e. To allow up to 200 sprays or applications. A typical container 3 as disclosed in WO 96/06011 A2 holds a volume of about 2 to 10 ml.

The container 3 is preferably of substantially cylindrical or cartridge-shaped construction and can be inserted into the atomizer 1 from below once the latter has been to opened, and is optionally replaceable. It is preferably of rigid construction while in particular the fluid 2 is held in a collapsible bag 4 in the container 3.

The atomizer 1 also comprises a conveying device, particularly a pressure generator 5 for delivering and atomizing the fluid 2, particularly in a predetermined or adjustable dosage amount.

The atomizer 1 or pressure generator 5 has in particular a holder 6 for the container 3, an associated drive spring 7 which is only partly shown, preferably with a locking element 8 which is manually operable to release it, a conveying tube 9 preferably in the form of a capillary with an optional valve, particularly a non-return valve 10, a pressure chamber 11 and/or an outlet nozzle 12 particularly in the region of a mouthpiece 13.

The container 3 is fixed, particularly by latching or locking, in the atomizer 1 via the holder 6 such that the conveying tube 9 dips into the container 3. The holder 6 may be embodied such that the container 3 can be replaced.

For axially tensioning the drive spring 7, the holder 6 with the container 3 and the conveying tube 9 is moved downwards in the drawings and fluid 2 is sucked out of the container 3 through the non-return valve 10 into the pressure chamber 11 of the pressure generator 5.

During the subsequent release of tension after actuation of the locking element 8, the fluid 2 in the pressure chamber 11 is put under pressure by moving the conveying tube 9 with its now closed non-return valve 10 back upwards, by releasing the drive spring 7, to act as a ram. This pressure expels the fluid 2 through the nozzle 12, during which time it is atomized into the aerosol 14 preferably destined for the lungs, as indicated in FIG. 1.

The user or patient (not shown) can inhale the aerosol 14, while a supply of air can to preferably be sucked into the mouthpiece 13 through at least one air inlet opening 15.

The atomizer 1 has in particular an upper housing part 16 and an inner part 17 which is rotatable relative thereto (FIG. 2) having an upper part 17a and a lower part 17b (FIG. 1), while an in particular manually operable housing part 18 is releasably attacked to, in particular pushed onto, the inner part 17, preferably by means of a holding element 19. For inserting and/or changing the container 3, the housing part 18 can be detached from the atomizer 1. The housing part 18 preferably forms a cap-like lower housing part and/or engages around or over a free lower end portion of the container 3.

The housing part 18 can be rotated relative to the upper housing part 16, carrying with it the portion 17b of the inner part 17 which is lower in the drawing. The drive spring 7 is thus tensioned in the axial direction via a gear (not shown) acting on the holder 6. During tensioning, the container 3 is moved axially downwards or moved with its end portion (further) into the housing part 18 or towards the end face thereof until the container 3 assumes an end position as shown in FIG. 2. In this position, the drive spring 7 is under tension.

When tension is applied for the first time the container 3 is preferably pierced or opened at the base. In particular, an axially acting spring 20 mounted in the housing part 18 comes to abut on the base 21 of the container, piercing the container 3 or a seal provided in its base with a piercing element 22 when contact is made for the first time, in order to ventilate it.

During the atomizing process, the container 3 is moved back into its original position by the drive spring 7.

The container 3 thus performs a lifting movement during the tensioning process and during the atomizing process.

To complete the disclosure of the present patent application, reference is hereby made, as a precaution, to the total disclosure of both WO 91/14468 A1 and WO 97/12687 A1. Generally, the disclosure therein preferably refers to a nebulizer having a spring pressure of 5 to 60 MPa, preferably 10 to 50 MPa on the fluid, with volumes per actuation of 10 to 50 μl, preferably 10 to 20 μl, most preferably about 15 μl. At the same time the fluid is converted into an aerosol, the droplets of which have an aerodynamic diameter of up to 20 μm, preferably 3 to 10 μm. Moreover, the disclosure therein preferably relates to a nebulizer with a cylinder-like shape which is about 9 cm to about 15 cm long and about 2 to about 5 cm wide and a nozzle spray spread of from 20° to 160°, preferably from 80° to 100°. These magnitudes also apply to the nebulizer according to the teaching of the invention as particularly preferred values.

The construction and mode of operation of several embodiments of the proposed atomizer 1 are explained in more detail hereinafter, referring to the other figures that are purely schematic and not to scale and drawing attention only to major differences from the atomizer according to FIGS. 1 and 2. The remarks made concerning FIGS. 1 and 2 thus apply or have a complementary value, while any desired combinations of features of the atomizer 1 according to FIGS. 1 and 2 and the atomizer 1 according to the embodiments described hereinafter or with one another are possible.

FIGS. 3 and 4 show in schematic section the proposed atomizer 1 according to a first embodiment. FIG. 3 shows the delivery condition with the container 3 closed and in particular sealed. FIG. 4 shows the activated state, i.e. with the container 3 already opened.

FIGS. 3 and 4 show the atomizer 1 with an optional cover 23, in particular in the form of a cap, which covers or closes off the mouthpiece 13 and in particular also the air inlet openings 15 when the atomizer 1 is not in use. The cover 23 can be pulled off, flipped open or removed or opened up in some other way to enable the atomizer 1 to to be used.

Preferably, the (still) closed container 3 is already installed in the atomizer 1 in its delivered state, as shown in FIG. 3. In the closed state, in the embodiments shown, an outer seal 24 at the head end of the container 3 and/or a septum 25, a membrane, a plastics closure or the like provided inside the container 3 has not yet been opened.

Moreover, in the embodiment shown, in the closed state, a vent opening 26 preferably provided in the base of the container 3, which can be opened by the piercing element 22, is closed, i.e. has not yet been pierced. It should be noted that depending on the particular design the container 3 may also have fewer and/or different opening options.

The atomizer 1 is preferably constructed so that the container 3 is or can be opened within the atomizer 1 before or during the first use of the atomizer 1. In particular, the container 3 is open when the seal 24 and the septum 25 or the like are both opened. This is also referred to here for short as the activated state. The piercing or opening of the vent opening 26 may take place separately, in particular at a later stage when the atomizer 1 is tensioned (for the first time).

The opening of the container 3 is carried out, as proposed, in particular by means of a conveying element, particularly the conveying tube 9 or the like, preferably by piercing the container 3 or inserting into the container 3. By a corresponding relative movement, particularly in the longitudinal direction or the direction of lifting of the container 3 relative to the conveying tube 9, the conveying tube 9 pierces the seal 24 and is inserted through the septum 25 into the interior of the container 3, particularly into the bag 4, thereby opening the container 3, namely forming a fluidic connection allowing the fluid 2 to escape from the container 3. The container 3 is thus opened in particular at its head end.

During the normal tensioning and atomizing strokes, the container 3 is preferably to moved together with the conveying element or conveying tube 9 by means of the holder 6, while the fluidic connection formed is maintained, i.e. the container 3 preferably remains open.

The venting, preferably at the base, mentioned above achieved by opening the vent opening 26 may take place before or during or after the opening of the container 3, particularly at its head end, depending on the embodiment or particular requirements.

In the first embodiment the container 3 is pre-installed and the housing part 18 has not been pushed up fully in the axial direction in the delivered state. Rather, between the housing part 18 and the upper housing part 16, a securing member 27 is provided, in particular, so that the housing part or lower part 18 is moved sufficiently far away from the upper housing part 16 in order to be able to hold the (still) closed container 3 axially away from the conveying tube 9.

In the non-activated, moved away state, the housing part 18 is preferably held by means of at least one latching arm 29 or the like arranged on the upper housing part 16 or inner part 17 so that it cannot be lost and in particular cannot be undone. Preferably the latching arm 28 engages with a latching lug 29 in a latching recess 30 in the housing part 18 and thus secures the housing part 18 by interlocking engagement against being axially pulled off completely. However, here again, different design solutions are also possible.

In particular, the housing part or lower part 18 of the atomizer 1 cannot be detached from the atomizer 1 after the first (partial) axial pushing up, i.e. the atomizer 1 cannot be opened again, with the result that the container 3 cannot be replaced, and in particular cannot be removed again.

The securing member 27 is essentially of hollow cylindrical construction, for example, and is arranged axially between the housing part 18 and the upper housing part 16. In order to be able to activate the atomizer 1, i.e. push the housing part 18 fully to upwards in the axial direction and thereby open the container 3, first of all the securing member 27 has to be removed or overcome. In particular, the securing member 27 is in the form of a banderole or the like, for example made of plastics, and can be manually opened, removed, overcome, broken, cut off or destroyed. The securing member 27 may alternatively or simultaneously form or constitute a tamper-evident seal. However, other embodiments of the securing member 27 are also possible, for example in the form of a securing tab or the like.

Instead of the securing member 27 any other suitable securing means may be used. For example, it is possible for the housing part 18 to have to be rotated initially to some extent in order to be pushed axially (fully) upwards. Reference is made in particular to WO 2006/125577 A1 regarding possible embodiments of the securing means, and this publication is hereby incorporated as a supplementary disclosure.

After or during the removal or overcoming of the securing member 27 or other securing means, a user (not shown) can push the housing part 18 fully upwards in the axial direction and thereby bring about the activated state of the atomizer 1, namely open the container 3 by inserting the conveying element or conveying tube 9. FIG. 4 shows this activated state with the housing part 18 pushed fully upwards. In this pushed up state the housing part 18 is preferably secured or held by interlocking engagement, particularly by the engagement of the latching arm 28 or latching lug 29 in a corresponding additional latching recess 31 or by other mechanical securing means.

FIG. 4 shows the atomizer 1 or container 3 in the activated state, in which the container 3 has already been opened and the housing part 18 is pushed fully upwards in the axial direction. In order to engage the holder 6 with the container 3 at the head end and then be able to move the container 3 for the tensioning and pressing movements, it may be necessary to tension the atomizer 1 for the first time. During this tensioning process the holder 6 together with the conveying tube 9 is moved axially towards or into the housing part 18, thereby bringing the holder 6 into engagement with the container 3 and preferably also pressing the container 3 against the piercing element 22 in the region of the base of the housing part 18 and thereby piercing or opening the vent opening 26. FIG. 4 shows the atomizer 1 in the released state, i.e. After the first atomization, in particular. The holder 6 engages with the container 3 and the conveying tube 9 is inserted fully into the container 3.

In the delivered state shown in FIG. 3, i.e. in which the container 3 is still sealed, the atomizer 1 may be stored. In particular, the closed seal 24 ensures that any solvent which has to be present in the fluid 2 cannot escape or can only escape in very small amounts.

In order to prevent unwanted opening of the container 3, particularly of the seal 24 or vent opening 26, in the delivered state of the atomizer 1, the atomizer 1 preferably has a transporting safety means (not shown). The transporting safety means prevents the container 3 from being undesirably moved axially in the atomizer 1, e.g. during transportation, during accidental falling of the atomizer 1 or the like, by a frictional or interlocking engagement, for example.

Possible methods of securing the container 3 in transport are disclosed in particular in WO 2006/125577 A1, which is introduced at this point as a supplementary disclosure.

It should be noted that the opening of the container 3 takes place preferably solely by mechanical action and/or mechanical actuation. However, in addition or alternatively, it is also possible to open it in some other way, for example chemically, electrically, magnetically, pneumatically, hydraulically or the like.

The proposed atomizer 1 is activated once the container 3 or housing part 18 has been fully pushed upward in the axial direction and can be used in the same way as the atomizer 1 shown in FIGS. 1 and 2.

In contrast to freestanding appliances or the like, the proposed inhaler 1 is preferably designed to be portable and in particular is a mobile hand-held device.

The proposed atomizer operates purely mechanically, in particular. However, the atomizer 1 may theoretically operate by any other method. In particular, the expression “conveying device” or “pressure generator” must be understood in very general terms. For example, the pressure required for the delivery and atomization may also be produced by propellant gas, a pump or any other suitable method.

The proposed atomizer 1 is designed in particular for the brief atomization of the fluid 2, for example for one to two breaths. However, it may also be designed or used for longer or continuous nebulisation.

The atomizer 1 preferably has a protective device 32 for preventing or at least reducing any possible contamination of the fluid 2 with germs.

In the first embodiment the protective device 32 has a germ-proof, particularly sterile wrapping, preferably a bag 33, to hold the atomizer 1 and preferably also the container 3 in the delivery state, as shown in FIG. 3. The wrapping or bag 33 is preferably made from foil or plastic material and/or is at least partially transparent. The wrapping or bag 33 is preferably of gas-tight construction.

The wrapping or bag 33 surrounds the atomizer 1 preferably relatively loosely and/or is particularly flexible or elastic, so that the atomizer or container 3 can preferably still be activated while the wrapping is sealed.

After activation, the wrapping can be opened, particularly torn or cut open, and removed, so that the atomizer can then be used normally. In FIG. 4 the bag 33 has already been removed.

The protective device 32 or wrapping means in particular that the atomizer 1 and container 3 can be stored for a very long time in a germ-proof and particularly sterile manner.

According to a first alternative embodiment the already sterile container 3 is inserted in the sterile atomizer 1 under sterile conditions and the atomizer 1 is then surrounded or enclosed by the equally sterile wrapper under sterile conditions. There is then no necessity for subsequent or additional sterilisation, although this is still possible.

According to a second alternative embodiment the sterilisation is only carried out after the container 3 has been placed in the atomizer 1 and optionally after the atomizer 1 has been enclosed in the wrapper. In this case the atomizer 1, container 3 and/or wrapper need not be sterilised beforehand and/or need not be handled under sterile conditions.

Sterilisation may be carried out in particular by irradiation (e.g. microwave, UV, X-ray or gamma-ray radiation) and/or using gas (e.g. ethylene oxide). In some cases it may be sufficient to fill the wrapping with a sterilising gas as it is sealed.

Moreover, the protective device 32 may alternatively or additionally comprise a germ-proof receiving space 34 for the container 3 in the atomizer 1. In particular, the receiving space 34 is formed by the atomizer 1, particularly by the housing part 18, which in this case can be fitted on in germ-proof manner. However, here, too, other design solutions are possible.

Sterilisation is then carried out as in the case of the bag 33, preferably before, during and/or after sealing.

According to another alternative embodiment the protective device 32 may have a contact section 35 for the fluid 2 which has the effect of killing bacteria or other germs or having an antibacterial activity. The contact section 35 is shown in FIG. 5, to which shows a schematic magnified cross-section through part of the atomizer 1, particularly an outlet or nozzle assembly 36.

The contact section 35 has an oligodynamic activity, in particular. Preferably, the contact section 35 contains silver and/or copper, alloys or mixtures thereof or the like or may be made from them. Alternatively or additionally, the contact section 35 may also contain activated charcoal or be made from it.

The contact section 35 is preferably arranged in a section of the fluid path, particularly in the outlet or nozzle assembly 36, in an outlet channel 37 or in the conveying device or the pressure generator 5 or in or on the container 3, or formed thereby.

Particularly preferably, the contact section 35 is arranged close to the exit nozzle 12 or some other outlet and/or downstream of the pressure chamber 11.

The contact section 35 may alternatively or additionally be arranged in the pressure chamber 11, if required. Alternatively or additionally, the contact section 35 may also be provided on the container 3, particularly on its outlet and/or on or in the conveying tube 9 or the like.

Theoretically, however, the contact section 35 may also be arranged at other suitable places. If required, a plurality of contact sections 35 may be provided along the fluid path.

In the embodiment shown, the contact section 35 forms, in particular, part of a wall of a channel, such as the outlet channel 37. For this purpose, the contact section 35 is of hollow cylindrical construction, in particular, or is provided with a corresponding bore, opening or the like. However, other design solutions are also possible. In particular, it is also possible for the contact section 35 to form a flat side or opposing flat sides of a very flat channel section, in order to achieve the largest possible contact area between the contact section 35 on the one hand and the fluid 2 on the other hand.

The contact section 35 may generally be formed by a component or a section of a component of the atomizer 1 or an at least partial coating.

Alternatively or additionally the contact section 35 may also be formed by a material mixture and/or in particular discrete particles or other, particularly oligodynamically or anti-bacterially effective particles or particles of this kind may be integrated in a material (e.g. metal, composite material, plastics or ceramics), so that the particles can interact with a fluid 2. Alternatively or additionally, the particles may also be arranged in a surface region or may form a coating. According to an alternative embodiment, the conveying element or conveying tube 9 is made from a corresponding anti-bacterially effective material such as an alloy that contains silver, and/or is coated therewith. Alternatively or additionally, plastics surfaces may be coated accordingly to form the contact section 35.

According to an alternative embodiment the contact section 35 is formed on or in the exit nozzle 12 or outlet or nozzle assembly 36.

Particularly preferably, the contact section 35 is formed or arranged in a residual droplet region on the outside of the nozzle 12 or outlet or nozzle assembly 36.

Alternatively or additionally, the contact section 35 may also be integrated in a filter or formed thereby.

According to a particularly preferred embodiment, all the metal components of the atomizer 1 that come into contact with the fluid 2 are at least substantially made from and/or coated with the same material and/or material with at least substantially the same electronegativity.

The outlet or nozzle assembly 36 preferably comprises the outlet channel 37 with the adjoining outlet nozzle 12. Particularly preferably, the outlet or nozzle assembly 36 is made in one piece—optionally with the exception of the contact section 35—, particularly preferably from ceramics, sintered material, silicon, glass or the like.

The outlet or nozzle assembly 36 is preferably fluidically connected to the pressure chamber 11 on the inlet side. In particular, it is installed inside the mouthpiece 13 so that when the fluid is dispensed the desired atomization of the fluid 2 in the form of an aerosol 14 takes place in and/or out of the mouthpiece 13. Alternatively or additionally, the cover 23 may also be constructed as a protective device 32. In this case, the cover 23 is preferably constructed so as to provide a germ-proof seal to prevent or at least reduce possible contamination of the fluid 2 through the outlet nozzle 12. If necessary the cover 23 may abut directly on the outlet nozzle 12 or the outlet or nozzle assembly 36 for this purpose. Alternatively or additionally, the cover 23 may be of oligodynamic construction particularly in this region or the outlet or nozzle assembly 36 may be of oligodynamic construction in this region or may comprise or form a contact section 35 as described above.

A second embodiment of the proposed atomizer 1 will now be described with reference to FIG. 6, while only the features that differ essentially from the first embodiment will be discussed. The remarks and explanations provided earlier therefore apply here accordingly or in a complementary capacity.

FIG. 6 shows, in a view corresponding to FIG. 5, a schematic section, again not to scale, through the outlet or nozzle assembly 36 of the atomizer 1 according to the second embodiment.

In the second embodiment the protective device 32 comprises additionally or alternatively to the options described above a lockable valve 38 for preventing or at least minimizing contamination of the fluid 2 in the container 3, in the conveying element such as the conveying tube 9, in the pressure chamber 11, in the outlet channel 37 and/or in another section carrying the fluid or generally in the conveying device or to pressure generator 5. The valve 38 blocks off an associated channel, such as the outlet channel 37, in particular, in order to prevent or at least minimize the entry of germs.

In the embodiment shown, the valve 38 is associated with the outlet or nozzle assembly 36 and more particularly is incorporated therein. However, other design solutions are also possible.

The valve 38 preferably closes automatically, particularly by spring force. For example, a moveable valve element 39 is biased by a closing spring 40 into the closed position shown in FIG. 6, in which it blocks the outlet channel 37.

The valve 38 can preferably be opened by the fluid pressure acting on it. In the embodiment shown, a connecting channel 41 is provided for this purpose which branches off from the outlet channel 37 upstream of the valve 38, in order to be able to open the valve element 39 counter to the force of the closing spring 40 under corresponding fluid pressure. However, here again, other design solutions are also possible.

In particular, the valve 38 may be in the form of a one-way or non-return valve.

In the embodiment shown, the valve 38 preferably operates purely mechanically or hydraulically. However, other design solutions are also possible. For example, the valve 38 may also operate and in particular be opened and/or closed electrically, electromagnetically, piezoelectrically, pneumatically or by some other suitable method.

The valve 38 is preferably designed to block the outlet channel 37 or some other channel section for conveying the fluid 2. However, according to an alternative embodiment (not shown) it may be sufficient to interrupt the fluid current or column of fluid, for example by means of a suitable capillary stop or the like, so that two separate fluid regions or columns are formed, for example, which are not directly in fluidic contact with one another, thus making it impossible or at least difficult for germs to be transferred between them.

The valve 38 like the contact section 35 may be arranged at different places. In particular, the valve 38 is arranged immediately adjacent to or upstream of the outlet nozzle 12 and/or downstream of the pressure chamber 11. Additionally or alternatively the valve 38 may also be arranged on the container 3, in particular in order to block its outlet.

If desired the contact section 35 may also be integrated in the valve 38, or vice versa.

The schematic section according to FIG. 7 corresponding to FIGS. 5 and 6 shows a third embodiment of the proposed atomizer 1. Again only the outlet or nozzle assembly 36 is shown. The remarks and explanations given above apply accordingly.

In the third embodiment, as the protective device 32, in addition to or instead of the options previously described, a filter 42 is provided which has such fine openings 43 that it is at least substantially germ-proof. Pathogens such as bacteria or the like therefore cannot for the most part pass through the openings 43.

The filter 42 is preferably arranged in the outlet channel 37 or another channel section, so that the fluid 2 has to pass through the filter 42 initially, in particular immediately before it is delivered through the exit nozzle 12 or some other outlet. The filter 42 can then in the same way as the contact section 35 or valve 38, prevent or at least minimize the unwanted penetration of germs at the outlet end or nozzle end.

In the embodiment shown, the filter 42 is arranged downstream of the pressure chamber 11 or conveying device or pump or the pressure generator 5 and/or upstream of the exit nozzle 12.

The atomizer 1 may also comprise a plurality of exit nozzles 12, as shown by way of example in FIG. 7. The exit nozzles 12 are then selectively connected via a common channel or—as shown—via separate outlet channels 37′ and 37″, in this case to the filter 42, in particular.

FIGS. 8 to 12 show a proposed filter 42 which can be used in particular as a protective device 32 I the proposed atomizer 1—particularly preferably in the third embodiment, particularly integrated in the outlet or nozzle assembly 36—or in other atomizers 1, inhalers or the like. The filter 42 is particularly intended or usable for preventing or at least minimizing the possible contamination of a fluid 2 which is preferably to be atomized. However, the fluid 2 may also be dispensed by any other suitable method.

FIG. 8 shows the open filter 42 in plan view. FIG. 9 shows a magnified detail of FIG. 8. FIG. 10 shows the closed filter 42 in schematic section along the line X-X in FIG. 8. FIG. 11 shows a magnified detail from FIG. 10. FIG. 12 shows, in perspective view, a detail of the filter 42 with openings 43 which are not covered.

The filter 42 has such fine openings 43 that it is at least substantially germ-proof. In particular, the median or maximum diameter d (FIG. 12) or a maximum cross-sectional extent of the openings 43 is at most 1 nm, particularly 0.5 nm, particularly preferably about 0.5 nm or less.

The openings 43 are preferably substantially rectangular in cross-section. The maximum or mean width b and/or the maximum or mean height h and/or the maximum diameter d of the openings 43 is preferably at most 1 nm, particularly at most 0.5 nm, particularly preferably at most 0.3 nm or less. In the embodiment shown, the width b and the height h are substantially 0.2 nm.

The openings 43 are preferably longer than their maximum diameter d. This contributes to the high stability and particularly the high pressure resistance of the filter 42. In particular, their length 1 is about 1 nm to 5 nm.

The filter 42 is preferably made from a piece of material 44 and a covering element 45. FIG. 8 shows the filter 42 or piece of material 44 in plan view without the covering element 45. The section shown in FIG. 10 shows the filter 42 or piece of material with the covering element 45.

The openings 43 are formed in a flat side of the piece of material 44, in the embodiment shown, as illustrated particularly in FIG. 10.

Preferably, the openings 43 are thus formed between the piece of material 44 and the covering element 45. However, other design solutions are also possible.

In the embodiment shown, the piece of material 44 is formed starting from a flat side and structured in the desired manner, particularly by etching, as known from semiconductor technology, for example, by punching, by laser machining and/or by any other suitable processing method. For example, larger areas may also be cut away using a milling machine. The machining accuracy is essential as very fine, defined openings 43 are formed.

In the embodiment shown, the openings 43 are preferably substantially located in one plane which runs parallel to the direction of flow through the openings 43.

The openings 43 are preferably laterally bounded or covered by a common cover, namely the covering element 45.

The openings 43 are preferably arranged in linear manner adjacent to one another and extend particularly in a meandering (FIG. 8) or zigzag configuration or in any other suitable manner.

In the embodiment shown the filter 42 preferably comprises an inlet 46 and an adjoining distribution chamber 47. A filter structure 48 having the openings 43 separates the distribution chamber 47 from a collecting chamber 49 located on the other side of the filter structure 48, which is connected to an outlet 50 of the filter 42.

The inlet 46 is preferably connected to the pressure chamber 11 or the outlet channel 37. The outlet 50 is preferably connected to the outlet channel 37 or the exit nozzle 12. In particular, the filter 42 is attached such that the fluid 2, not shown in FIGS. 8 to 12, is forced to pass through the filter 42 as it exits, i.e. Is forced to flow through the openings 43.

In the embodiment shown, the openings 43, the inlet 46, the distribution chamber 47, the collecting chamber 49 and/or the outlet 50 are preferably formed as depressions in the piece of material 44 and in particular are surrounded by a standing edge of the piece of material 44 through which the connection to the covering element 45 is made.

The filter structure 48 extends in particular in a substantially meandering, zigzag shaped, linear configuration or any other suitable manner, particularly preferably in a manner such that as many openings 43 as possible can be formed.

In the embodiment shown, the filter 42 comprises, for example, more than 1,000 openings 43 with a relatively low total volume and in particular a very small volume of fluid 2 (preferably about 1 μl or less).

The plurality of parallel connected openings 43 leads to a flow resistance which is comparatively or at least sufficiently low.

In order to achieve a high stability of the filter structure 48 with the openings 43, the filter structure 48 initially has a preferably rib-like or strip-like elevation 51 which rises up from the base of the distribution chamber 47 or collecting chamber 49 towards the covering element 45. The elevation 51 has a width B of a few nm, for example, approximately 5 nm in the embodiment shown. The elevation 51 is in particular of continuous and/or linear configuration and extends in particular in a meandering or zigzag shape, in a linear configuration or the like in accordance with the filter structure 48.

Arranged on the elevation 51 is a preferably narrower raised portion 52 in particular in the form of a web, rib or strip, which extends along the elevation 51 and has for example a width which is reduced to about 5 to 50% compared with the elevation 51.

The height and width of the raised portion 52 is about 1 nm in each case, for example. Depending on the desired length of the openings 43 the width may also be up to 5 nm, for example. The same applies to heights for possibly varying the depths or cross-section of the openings 43.

Starting from the flat side of the raised portion 52 the openings 43 are formed by, in particular, channels or groove-like recesses or depressions in the raised portion 52.

The mean spacing of the openings 43 is about 1 nm, for example, or the width or thickness of the raised portion 52. However, other sizes are also possible.

In the representation according to FIG. 8 the flat side of the piece of material 44 and the flat side of the raised portion 52 face the onlooker. The openings 43 are still open on the flat side—i.e. the longitudinal side—and are preferably only covered and sealed off at the sides by the covering element 45 or some other covering. This makes it substantially easier to manufacture with a high degree of precision or low tolerances, particularly by etching. However, other design solutions are also possible.

In order to improve the particular connection to the covering element 45 still further and/or further increase the stability of the filter structure 48 in the filter 42, particularly with regard to an especially high pressure stability and other strength of the filter 42, support structures 53 may optionally be provided, as indicated in FIG. 8. The support structures 53 extend from the elevation 51 alongside the raised portion 52 towards the covering element 45, to which they may preferably also be connected. In FIG. 10 the optional support structures 53 are not shown, in the interests of simplicity.

The starting material used for the piece of material 44 is preferably solid or sheet material.

The piece of material 44 preferably consists of silicon or some other suitable material, e.g. sintered material, ceramics, glass or the like. The covering element 45 consists in particular of the same material or another suitable material, preferably glass. The material 44 and the covering element 45 are joined together in particular by so-called bonding or welding. However, basically any other suitable method of attachment or a sandwich construction or the like are also possible here.

In a particularly preferred alternative embodiment, a piece of sheet material (not shown), particularly a silicon wafer, is used from which a plurality of pieces of material 44 are produced for a plurality of filters 42. Before the material is broken down into individual pieces 44 or filters 42, preferably the structures, especially depressions, are first produced starting from a flat side of the piece of sheet material for the plurality of pieces of material 44. This is done in particular by means of the production or etching of fine structures which is conventional in semiconductor manufacture, and consequently reference may be made to the prior art for the etching of silicon or the like in this respect.

Particularly preferably, the covering element 45, like the piece of material 44, is produced from a sheet which is broken down or divided into a plurality of covering elements 45. For manufacturing the piece of material 44, a silicon wafer is preferably used in the form of a sheet, as already explained. A silicon wafer or some other wafer, a sheet of glass or the like may also be used for the sheet material for producing the covering elements 45.

If a piece of sheet material is used both for the manufacture of the pieces of material 44 and for the manufacture of the covering elements 45, the pieces of sheet are preferably joined together before they are broken down into the individual pieces of material 44 or covering elements 45. This makes assembly and positioning considerably easier.

The filter 42 may if necessary also comprise several—i.e. two or more—rows of openings 43. The rows may for example extend parallel to one another. For this purpose, several, i.e. two or more, raised portions 52 may be provided on an elevation 51, particularly running parallel and/or at a spacing from one another. Alternatively or additionally, several, particularly two or more, elevations 51 each having at least one raised portion 52 may be arranged behind one another or in series, particularly running parallel and/or at a spacing from one another.

Theoretically, the filter 42 may also be connected in combination, i.e. particularly in series, with another filter, such as a preliminary filter or the like.

The proposed filter 42 may also be used for the outlet or nozzle assembly 36, for example according to the third embodiment in FIG. 7.

In particular, it is possible to construct the filter 42 as a unit with the exit nozzle 12 or the outlet or nozzle assembly 36. This makes manufacture and assembly considerably easier. Particularly preferably the manufacture of the exit nozzle 12 or possibly a plurality of exit nozzles 12 takes place together with the associated filter 42, most preferably in the manner described above, using the piece of material 44 and the covering element 45, or any other suitable method.

The proposed solution may, however, be used not only in the atomizers 1 specifically described here but also in other atomizers or inhalers, related containers 3 or the like.

In particular, the proposed filter 42 may also be used for other purposes, for example in the purification of the fluid 2 and generally in the separation of germs, bacteria, cells or the like. In particular, the filter 42 is suitable and designed for small or tiny amounts in the μl range.

Generally, the atomizer 1 may also comprise another filter 42, e.g. of porous material, for protecting the exit nozzle 12 or the outlet or nozzle assembly 36 or the outlet channel 37 from possible clogging.

Individual features, aspects or properties of the various embodiments may also be combined with one another as desired or used in other atomizers, inhalers or the like.

The fluid 2 preferably contains ethanol and in particular contains ethanol as solvent.

Alternatively or additionally, the fluid 2 may contain EDTA (ethylenediaminetetraacetic acid) or the salts thereof as complexing agents.

Preferably, the fluid 2 is a liquid, as already mentioned, more particularly an aqueous, ethanolic or aqueous/ethanolic medicament preparation. However, it may also be a different medicament preparation, a suspension or the like.

The following are preferred compounds, ingredients and/or formulations of the preferably medical fluid 2. As already mentioned, they may be aqueous or non-aqueous solutions, mixtures, ethanol-containing or solvent-free formulations or the like. Particularly preferred are:

The compounds listed below may be used in the device according to the invention on their own or in combination. In the compounds mentioned below, W is a pharmacologically active substance and is selected (for example) from among the betamimetics, anticholinergics, corticosteroids, PDE4-inhibitors, LTD4-antagonists, EGFR-inhibitors, dopamine agonists, H1-antihistamines, PAF-antagonists and PI3-kinase inhibitors. Moreover, double or triple combinations of W may be combined and used in the device according to the invention. Combinations of W might be, for example:
W denotes a betamimetic, combined with an anticholinergic, corticosteroid, PDE4-inhibitor, EGFR-inhibitor or LTD4-antagonist,
W denotes an anticholinergic, combined with a betamimetic, corticosteroid, PDE4-inhibitor, EGFR-inhibitor or LTD4-antagonist,
W denotes a corticosteroid, combined with a PDE4-inhibitor, EGFR-inhibitor or LTD4-antagonist
W denotes a PDE4-inhibitor, combined with an EGFR-inhibitor or LTD4-antagonist
W denotes an EGFR-inhibitor, combined with an LTD4-antagonist.

The compounds used as betamimetics are preferably compounds selected from among albuterol, arformoterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmefamol, salmeterol, soterenol, sulphonterol, terbutaline, tiaramide, tolubuterol, zinterol, CHF-1035, HOKU-81, KUL-1248 and
3-(4-{6-[2-hydroxy-2-(4-hydroxy-3-hydroxymethyl-phenyl)-ethylamino]-hexyloxy}-butyl)-benzyl-sulphonamide
5-[2-(5,6-diethyl-indan-2-ylamino)-1-hydroxy-ethyl]-8-hydroxy-1H-quinolin-2-one
4-hydroxy-7-[2-{[2-{[3-(2-phenylethoxy)propyl]sulphonyl}ethyl]-amino}ethyl]-2(3H)-benzothiazolone
1-(2-fluoro-4-hydroxyphenyl)-2-[4-(1-benzimidazolyl)-2-methyl-2-butylamino]ethanol
1-[3-(4-methoxybenzyl-amino)-4-hydroxyphenyl]-2-[4-(1-benzimidazolyl)-2-methyl-2-butylamino]ethanol
1-[2H-5-hydroxy-3-oxo-4H-1,4-benzoxazin-8-yl]-2-[3-(4-N,N-dimethylaminophenyl)-2-methyl-2-propylamino]ethanol
1-[2H-5-hydroxy-3-oxo-4H-1,4-benzoxazin-8-yl]-2-[3-(4-methoxyphenyl)-2-methyl-2-propylamino]ethanol
1-[2H-5-hydroxy-3-oxo-4H-1,4-benzoxazin-8-yl]-2-[3-(4-n-butyloxyphenyl)-2-methyl-2-propylamino]ethanol
1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol
5-hydroxy-8-(1-hydroxy-2-isopropylaminobutyl)-2H-1,4-benzoxazin-3-(4H)-one
1-(4-amino-3-chloro-5-trifluoromethylphenyl)-2-tert.-butylamino)ethanol
6-hydroxy-8-{1-hydroxy-2-[2-(4-methoxy-phenyl)-1,1-dimethyl-ethylamino]-ethyl}-4H-benzo[1,4]oxazin-3-one
6-hydroxy-8-{1-hydroxy-2-[2-(ethyl 4-phenoxy-acetate)-1,1-dimethyl-ethylamino]-ethyl}-4H-benzo[1,4]oxazin-3-one
6-hydroxy-8-{1-hydroxy-2-[2-(4-phenoxy-acetic acid)-1,1-dimethyl-ethylamino]-ethyl}-4H-benzo[1,4]oxazin-3-one
8-{2-[1,1-dimethyl-2-(2,4,6-trimethylphenyl)-ethylamino]-1-hydroxy-ethyl}-6-hydroxy-4H-benzo[1,4]oxazin-3-one
6-hydroxy-8-{1-hydroxy-2-[2-(4-hydroxy-phenyl)-1,1-dimethyl-ethylamino]-ethyl}-4H-benzo[1,4]oxazin-3-one
6-hydroxy-8-{1-hydroxy-2-[2-(4-isopropyl-phenyl)-1.1-dimethyl-ethylamino]-ethyl}-4H-benzo[1,4]oxazin-3-one
8-{2-[2-(4-ethyl-phenyl)-1,1-dimethyl-ethylamino]-1-hydroxy-ethyl}-6-hydroxy-4H-benzo[1,4]oxazin-3-one
8-{2-[2-(4-ethoxy-phenyl)-1,1-dimethyl-ethylamino]-1-hydroxy-ethyl}-6-hydroxy-4H-benzo[1,4]oxazin-3-one
4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid
8-{2-[2-(3.4-difluoro-phenyl)-1,1-dimethyl-ethylamino]-1-hydroxy-ethyl}-6-hydroxy-4H-benzo[1,4]oxazin-3-one
1-(4-ethoxy-carbonylamino-3-cyano-5-fluorophenyl)-2-(tert-butylamino)ethanol
2-hydroxy-5-(1-hydroxy-2-{2-[4-(2-hydroxy-2-phenyl-ethylamino)-phenyl]-ethylamino}-ethyl)-benzaldehyde
N-[2-hydroxy-5-(1-hydroxy-2-{2-[4-(2-hydroxy-2-phenyl-ethylamino)-phenyl]-ethylamino}-ethyl)-phenyl]-formamide
8-hydroxy-5-(1-hydroxy-2-{2-[4-(6-methoxy-biphenyl-3-ylamino)-phenyl]-ethylamino}-ethyl)-1H-quinolin-2-one
8-hydroxy-5-[1-hydroxy-2-(6-phenethylamino-hexylamino)-ethyl]-1H-quinolin-2-one
5-[2(2-{4-[4-(2-amino-2-methyl-propoxy)-phenylamino]-phenyl}-ethylamino)-1-hydroxy-ethyl]-8-hydroxy-1H-quinolin-2-one
[3-(4-{6-[2-hydroxy-2-(4-hydroxy-3-hydroxymethyl-phenyl)-ethylamino]-hexyloxy}-butyl)-5-methyl-phenyl]-urea
4-(2-{6-[2-(2,6-dichloro-benzyloxy)-ethoxy]-hexylamino}-1-hydroxy-ethyl)-2-hydroxymethyl-phenol
3-(4-{6-[2-hydroxy-2-(4-hydroxy-3-hydroxymethyl-phenyl)-ethylamino]-hexyloxy}-butyl)-benzyl sulphonamide
3-(3-{7-[2-hydroxy-2-(4-hydroxy-3-hydroxymethyl-phenyl)-ethylamino]-heptyloxy}-propyl)-benzylsulphonamide
4-(2-{6-[4-(3-cyclopentanesulphonyl-phenyl)-butoxy]-hexylamino}-1-hydroxy-ethyl)-2-hydroxymethyl-phenol
N-Adamantan-2-yl-2-(3-{2-[2-hydroxy-2-(4-hydroxy-3-hydroxymethyl-phenyl)-ethylamino]-propyl}-phenyl)-acetamide
   optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The anticholinergics used are preferably compounds selected from among the tiotropium salts, preferably the bromide salt, oxitropium salts, preferably the bromide salt, flutropium salts, preferably the bromide salt, ipratropium salts, preferably the bromide salt, glycopyrronium salts, preferably the bromide salt, trospium salts, to preferably the chloride salt, tolterodine. In the above-mentioned salts the cations are the pharmacologically active constituents. As anions the above-mentioned salts may preferably contain the chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts the chlorides, bromides, iodides and methanesulphonates are particularly preferred.

Other preferred anticholinergics are selected from among the salts of formula AC-1

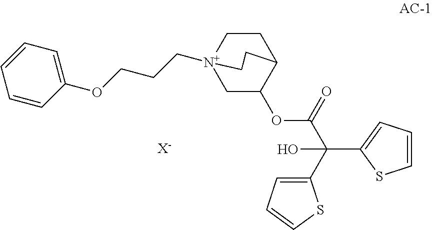

wherein X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate, preferably an anion with a single negative charge, particularly preferably an anion selected from among the fluoride, chloride, bromide, methanesulphonate and p-toluenesulphonate, particularly preferably bromide, optionally in the form of the racemates, enantiomers or hydrates thereof. Of particular importance are those pharmaceutical combinations which contain the enantiomers of formula AC-1-en

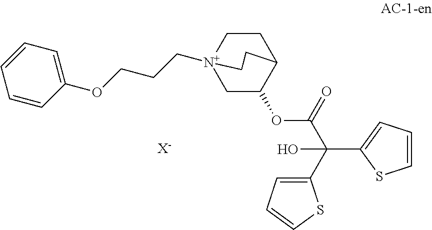

wherein X⁻ may have the above-mentioned meanings. Other preferred anticholinergics are selected from the salts of formula AC-2

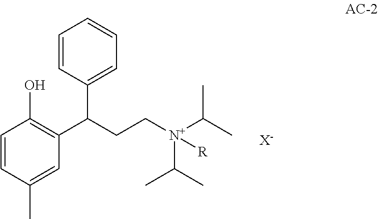

wherein R denotes either methyl or ethyl and wherein X⁻ may have the above-mentioned meanings. In an alternative embodiment the compound of formula AC-2 may also be present in the form of the free base AC-2-base.

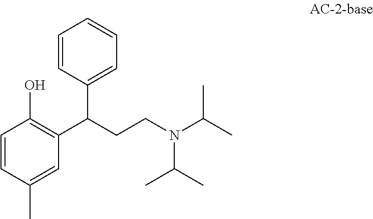

Other specified compounds are:
tropenol 2,2-diphenylpropionate methobromide,
scopine 2,2-diphenylpropionate methobromide,
scopine 2-fluoro-2,2-diphenylacetate methobromide,
tropenol 2-fluoro-2,2-diphenylacetate methobromide;
tropenol 3,3′,4,4′-tetrafluorobenzilate methobromide,
scopine 3,3′,4,4′-tetrafluorobenzilate methobromide,
tropenol 4,4′-difluorobenzilate methobromide,
scopine 4,4′-difluorobenzilate methobromide,
tropenol 3,3′-difluorobenzilate methobromide,
scopine 3,3′-difluorobenzilate methobromide;
tropenol 9-hydroxy-fluorene-9-carboxylate methobromide;
tropenol 9-fluoro-fluorene-9-carboxylate methobromide;
scopine 9-hydroxy-fluorene-9-carboxylate methobromide;
scopine 9-fluoro-fluorene-9-carboxylate methobromide;
tropenol 9-methyl-fluorene-9-carboxylate methobromide;
scopine 9-methyl-fluorene-9-carboxylate methobromide;
cyclopropyltropine benzilate methobromide;
cyclopropyltropine 2,2-diphenylpropionate methobromide;
cyclopropyltropine 9-hydroxy-xanthene-9-carboxylate methobromide;
cyclopropyltropine 9-methyl-fluorene-9-carboxylate methobromide;
cyclopropyltropine 9-methyl-xanthene-9-carboxylate methobromide;
cyclopropyltropine 9-hydroxy-fluorene-9-carboxylate methobromide;
cyclopropyltropine methyl 4,4′-difluorobenzilate methobromide.
tropenol 9-hydroxy-xanthene-9-carboxylate methobromide;
scopine 9-hydroxy-xanthene-9-carboxylate methobromide;
tropenol 9-methyl-xanthene-9-carboxylate methobromide;
scopine 9-methyl-xanthene-9-carboxylate methobromide;
tropenol 9-ethyl-xanthene-9-carboxylate methobromide;
tropenol 9-difluoromethyl-xanthene-9-carboxylate methobromide;
scopine 9-hydroxymethyl-xanthene-9-carboxylate methobromide,

The above-mentioned compounds may also be used as salts within the scope of the present invention, wherein instead of the methobromide the metho-X salts are used, wherein X may have the meanings given hereinbefore for X.

As corticosteroids it is preferable to use compounds selected from among beclomethasone, betamethasone, budesonide, butixocort, ciclesonide, deflazacort, dexamethasone, etiprednol, flunisolide, fluticasone, loteprednol, mometasone, prednisolone, prednisone, rofleponide, triamcinolone, RPR-106541, NS-126, ST-26 and
(S)-fluoromethyl 6,9-difluoro-17-[(2-furanylcarbonyl)oxy]-11-hydroxy-16-methyl-3-oxo-androsta-1,4-diene-17-carbothionate
(S)-(2-oxo-tetrahydro-furan-3S-yl)6,9-difluoro-11-hydroxy-16-methyl-3-oxo-17-propionyloxy-androsta-1,4-diene-17-carbothionate,
cyanomethyl 6α,9α-difluoro-11β-hydroxy-16α-methyl-3-oxo-17α-(2,2,3,3-tertamethylcyclopropylcarbonyl)oxy-androsta-1,4-diene-17β-carboxylate
   optionally in the form of the racemates, enantiomers or diastereomers thereof and optionally in the form of the salts and derivatives thereof, the solvates and/or hydrates thereof. Any reference to steroids includes a reference to any salts or derivatives, hydrates or solvates thereof which may exist. Examples of possible salts and derivatives of the steroids may be: alkali metal salts, such as for example sodium or potassium salts, sulphobenzoates, phosphates, isonicotinates, acetates, dichloroacetates, propionates, dihydrogen phosphates, palmitates, pivalates or furoates.

PDE4-inhibitors which may be used are preferably compounds selected from among enprofyllin, theophyllin, roflumilast, ariflo (cilomilast), tofimilast, pumafentrin, lirimilast, arofyllin, atizoram, D-4418, Bay-198004, BY343, CP-325.366, D-4396 (Sch-351591), AWD-12-281 (GW-842470), NCS-613, CDP-840, D-4418, PD-168787, T-440, T-2585, V-11294A, CI-1018, CDC-801, CDC-3052, D-22888, YM-58997, Z-15370 and
N-(3,5-dichloro-1-oxo-pyridin-4-yl)-4-difluoromethoxy-3-cyclopropylmethoxybenzamide
(−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide
(R)-(+)-1-(4-bromobenzyl)-4-[(3-cyclopentyloxy)-4-methoxyphenyl]-2-pyrrolidone
3-(cyclopentyloxy-4-methoxyphenyl)-1-(4-N′-[N-2-cyano-5-methyl-isothioureido]benzyl)-2-pyrrolidone
cis[4-cyano-4-(3-cyclopentyloxy-4-methoxyphenyl)cyclohexane-1-carboxylic acid]
2-carbomethoxy-4-cyano-4-(3-cyclopropylmethoxy-4-difluoromethoxy-phenyl)cyclohexan-1-one
cis[4-cyano-4-(3-cyclopropylmethoxy-4-difluoromethoxyphenyl)cyclohexan-1-ol]
(R)-(+)-ethyl[4-(3-cyclopentyloxy-4-methoxyphenyl)pyrrolidin-2-ylidene]acetate
(S)-(−)-ethyl[4-(3-cyclopentyloxy-4-methoxyphenyl)pyrrolidin-2-ylidene]acetate
9-cyclopentyl-5,6-dihydro-7-ethyl-3-(2-thienyl)-9H-pyrazolo[3,4-c]-1,2,4-triazolo[4,3-a]pyridine
9-cyclopentyl-5,6-dihydro-7-ethyl-3-(tert-butyl)-9H-pyrazolo[3,4-c]-1,2,4-triazolo[4,3-a]pyridine
   optionally in the form of the racemates, enantiomers or diastereomers thereof and optionally in the form of the pharmacologically acceptable acid addition salts thereof, the solvates and/or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The LTD4-antagonists used are preferably compounds selected from among montelukast, pranlukast, zafirlukast, MCC-847 (ZD-3523), MN-001, MEN-91507 (LM-1507), VUF-5078, VUF-K-8707, L-733321 and
1-(((R)-(3-(2-(6,7-difluoro-2-quinolinyl)ethenyl)phenyl)-3-(2-(2-hydroxy-2-propyl)phenyl)thio)methylcyclopropane-acetic acid,
1-(((1(R)-3(3-(2-(2,3-dichlorothieno[3,2-b]pyridin-5-yl)-(E)-ethenyl)phenyl)-3-(2-(1-hydroxy-1-methylethyl)phenyl)propyl)thio)methyl)cyclopropaneacetic acid
[2-[[2-(4-tert-butyl-2-thiazolyl)-5-benzofuranyl]oxymethyl]phenyl]acetic acid
   optionally in the form of the racemates, enantiomers or diastereomers thereof and optionally in the form of the pharmacologically acceptable acid addition salts, solvates and/or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate. By salts or derivatives which the LTD4-antagonists may optionally be capable of forming are meant, for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The dopamine agonists used are preferably compounds selected from among bromocriptin, cabergoline, alpha-dihydroergocryptine, lisuride, pergolide, pramipexol, roxindol, ropinirol, talipexol, tergurid and viozan, optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydrooxalate, hydrosuccinate, hydrobenzoate and hydro-p-toluenesulphonate.

H1-Antihistamines which may be used are preferably compounds selected from among epinastine, cetirizine, azelastine, fexofenadine, levocabastine, loratadine, mizolastine, ketotifen, emedastine, dimetindene, clemastine, bamipine, cexchlorpheniramine, pheniramine, doxylamine, chlorophenoxamine, dimenhydrinate, diphenhydramine, promethazine, ebastine, desloratidine and meclozine, optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

In addition, inhalable macromolecules as disclosed in EP 1 003 478 A1 or CA 2297174 A1 may also be used.

In addition, the compound may be selected from among the ergot alkaloid derivatives, the triptans, the CGRP-inhibitors, the phosphodiesterase-V inhibitors, optionally in the form of the racemates, enantiomers or diastereomers thereof, optionally in the form of the pharmacologically acceptable acid addition salts, the solvates and/or hydrates thereof.

Examples of ergot alkaloid derivatives are dihydroergotamine and ergotamine.

### LIST OF REFERENCE NUMERALS

| | |
|---|---|
| 1 | atomizer |
| 2 | fluid |
| 3 | container |
| 4 | bag |
| 5 | pressure generator |
| 6 | holder |
| 7 | drive spring |
| 8 | locking element |
| 9 | conveying tube |
| 10 | non-return valve |
| 11 | pressure chamber |
| 12 | exit nozzle |
| 13 | mouthpiece |
| 14 | aerosol |
| 15 | air inlet opening |
| 16 | upper housing part |
| 17 | inner part |
| 17a | upper portion of inner part |
| 17b | lower portion of inner part |
| 18 | housing part (lower part) |
| 19 | holding element |
| 20 | spring (in lower housing part) |
| 21 | container base |
| 22 | piercing element |
| 23 | cover |
| 24 | seal |
| 25 | septum |
| 26 | vent opening |
| 27 | securing member |
| 28 | latching arm |
| 29 | latching lug |
| 30 | latching recess |
| 31 | latching recess |
| 32 | protective device |
| 33 | bag |
| 34 | receiving space |
| 35 | contact section |
| 36 | outlet or nozzle assembly |
| 37 | outlet channel |
| 38 | valve |
| 39 | valve element |
| 40 | closing spring |
| 41 | connecting channel |
| 42 | filter |
| 43 | opening |
| 44 | piece of material |
| 45 | covering element |
| 46 | inlet |
| 47 | distribution chamber |
| 48 | filter structure |
| 49 | collecting chamber |
| 50 | outlet |
| 51 | elevation |
| 52 | raised portion |
| 53 | support structure |
| b | width (opening) |
| h | height (opening) |
| d | diameter (opening) |
| l | length (opening) |
| B | width (raised portion) |

1-36. (canceled)

37. Atomizer (1) in the form of an inhaler for medical aerosol therapy, for atomizing a fluid (2) in the form of a container (3) holding the fluid (2), characterised in that the atomizer (1) comprises a protective device (32) for preventing contamination of the fluid (2) with germs and/or in that the fluid (2) is free from preservatives.

38. Atomizer according to claim 37, characterised in that the container (3) contains several doses of the fluid (2) in its delivered state.

39. Atomizer according to claim 37, characterised in that the protective device (32) comprises a germ-proof sterile wrapping in the form of a bag (33), for holding the atomizer (1) and the container (3) in the delivered state.

40. Atomizer according to claim 39, characterised in that when the wrapper is sealed the container (3) can be opened, placed in the atomizer (1) and/or attached thereto.

41. Atomizer according to claim 37, characterised in that the atomizer (1) forms or comprises a germ-proof, particularly sterile receiving space (34) for the container (3) in the form of a housing part (18) that can be fitted on in germ-proof manner.

42. Atomizer according to claim 37, characterised in that in the delivered state the container (3) has already been inserted in the atomizer (1), but has not yet been sealed in germ-proof manner.

43. Atomizer according to claim 37, characterised in that in the delivered state of the atomizer (1) the sealed container (3) has already been arranged in the atomizer (1) and the atomizer (1) is configured such that the container (3) is opened inside the atomizer (1) before or during the first use of the atomizer (1).

44. Atomizer according to claim 37, characterised in that the atomizer (1) is constructed such that the container (3) cannot be exchanged, and in particular cannot be removed.

45. Atomizer according to claim 37, characterised in that the container (3) is movable in a lifting movement during the conveying of fluid, pressure generation and/or atomization.

46. Atomizer according to claim 37, characterised in that the atomizer (1) comprises a conveying element in the form of a conveying tube (9), for conveying the fluid (2) from the container (3).

47. Atomizer according to claim 37, characterised in that the atomizer (1) comprises a conveying device in the form of a pressure generator (5) having a conveying element in the form of a conveying tube (9), for conveying and/or atomizing the fluid (2).

48. Atomizer according to claim 47, characterised in that the container (3) can be opened by means of the conveying element by piercing or insertion.

49. Atomizer according to claim 37, characterised in that the protective device (32) has a contact section (35) for the fluid (2) with the effect of killing bacteria or other germs and/or with an antibacterial or oligodynamic effect.

50. Atomizer according to claim 49, characterised in that the contact section (35) contains silver, copper, an alloy silver and/or copper, or any combination thereof.

51. Atomizer according to claim 49, characterised in that the contact section (35) contains activated charcoal.

52. Filter (42) for a fluid (2) that is to be dispensed or purified for medical aerosol therapy, characterised in that the filter (42) has openings (43) that are fine enough to provide a filed that is at least substantially germ-proof.

53. Filter according to claim 52, characterised in that the openings (43) are substantially rectangular in cross-section.

54. Filter according to claim 52, characterised in that the maximum width (b), the maximum height (h) and/or the maximum diameter (d) of the openings (43) is at most 1 μm.

55. Method of producing an atomizer (1) according to claim 37, wherein:
(a) a germ-proof, sterile bag (33) is used as the protective device (32), the sterile container (3) is inserted under sterile conditions in the sterile atomizer (1) and the atomizer (1) is surrounded by the bag (33) under sterile conditions, or
(b) a germ-proof bag (33) is used as the protective device (32), the container (3) is inserted in the atomizer (1), the atomizer (1) with the container (3) is enclosed in the bag (33) and the bag (33) is sterilised with the atomizer (1) and container (3) by irradiation or gas.

56. Method of producing an atomizer (1) according to claim 37, wherein:
(a) a germ-proof, sterile housing part (18) is used as the protective device (32), the sterile container (3) is inserted in the sterile atomizer (1) and the container (3) in the atomizer (1) is covered by the housing part (18) under sterile conditions in germ-proof manner, or
(b) a germ-proof housing part (18) is used as the protective device (32), the container (3) in the atomizer (1) is covered by the housing part (18) in germ-proof manner and the atomizer (1) with the container (3) is then sterilised by radiation or gas.
